(19) United States
(12) Patent Application Publication
(10) Pub. No.: US 2026/0263288 A1
(43) Pub. Date: Sep. 10, 2026

(54) Nasal Drip Absorbing Device

(71) Applicants: Kurt Engel, Saint Charles, MO (US); Jami Taylor, Saint Charles, MO (US)

(72) Inventors: Kurt Engel, Saint Charles, MO (US); Jami Taylor, Saint Charles, MO (US)

(21) Appl. No.: 19/069,888

(22) Filed: Mar. 4, 2025

(51) Int. Cl. A61F 13/20

(52) U.S. Cl. CPC A61F 13/2005

(57) ABSTRACT

A nasal drip absorbing device for absorbing mucous from a nose of a user includes a nose clip that has a medial portion and a pair of leg portions. The pair of leg portions can be inserted into a respective nostril of the nose of the user having the medial portion extending across a septum of the nose of the user. A pair of cylindrical pads is each disposed on a respective one of the leg portions. Each cylindrical pad is positioned within a respective nostril of the nose of the user when the pair of leg portions is inserted into the respective nostril. Each cylindrical pad is comprised of a fluid absorbent material to absorb the mucous when each cylindrical pad is positioned within the respective nostril.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to nasal drip devices and more particularly pertains to a new nasal drip device for absorbing mucous in a nose of a user when the user has a nasal infection. The device includes a nose clip that has a medial section extending between a pair of leg sections and each of the leg sections can be inserted into a respective nostril of a nose of a user. The device includes a pair of cylindrical pads and each cylindrical pad is coupled to a respective leg section of the nose clip. Each cylindrical pad is positioned in a respective nostril when the leg portions of the nose clip are inserted into the respective nostrils to absorb the mucous in the nose of the user.

### (2) Description of Related Art Including Information Disclosed Under 37 CFR 1.97 and 1.98

The prior art relates to nasal drip devices including: a nose bleed clip device which includes a U-shaped clip and a pair of ovoid pads attached to the clip which are each insertable into respective nostrils of a user to absorb blood in the respective nostrils and a pair of stops attached to the U-shaped clip to inhibit the U-shaped clip from being inserted too far into the nostrils; a nasal depository device which includes an absorbent member that is insertable into a nostril of a user which has a hollow portion to enable the user to breathe through the hollow portion; a endonasal filter device which includes a curved clip and a pair of planar filters attached to the curved clip which are each insertable into a respective nostril of a user to filter particulates when the user breathes through their nostrils. In no instance does the prior art disclose a nasal drip absorbing device that includes a nose clip which is U-shaped and a pair of cylindrical pads each coupled to the nose clip which are each insertable into a respective nostril of a user to absorb mucous in the respective nostril when the user has a nasal infection.

### BRIEF SUMMARY OF THE INVENTION

An embodiment of the disclosure meets the needs presented above by generally comprising a nose clip that has a medial portion and a pair of leg portions. The pair of leg portions can be inserted into a respective nostril of the nose of the user having the medial portion extending across a septum of the nose of the user. A pair of cylindrical pads is each disposed on a respective one of the leg portions. Each cylindrical pad is positioned within a respective nostril of the nose of the user when the pair of leg portions is inserted into the respective nostril. Each cylindrical pad is comprised of a fluid absorbent material to absorb the mucous when each cylindrical pad is positioned within the respective nostril.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a nasal drip absorbing device according to an embodiment of the disclosure.

FIG. 2 is a perspective in-use view of an embodiment of the disclosure showing a nose clip being positioned for insertion into nostrils of a nose of a user.

FIG. 3 is a perspective in-use view of an embodiment of the disclosure showing a nose clip being inserted into nostrils of a nose of a user.

FIG. 4 is a perspective in-use view of an embodiment of the disclosure showing a nose clip being retained in nostrils of a nose of a user.

### DETAILED DESCRIPTION OF THE INVENTION

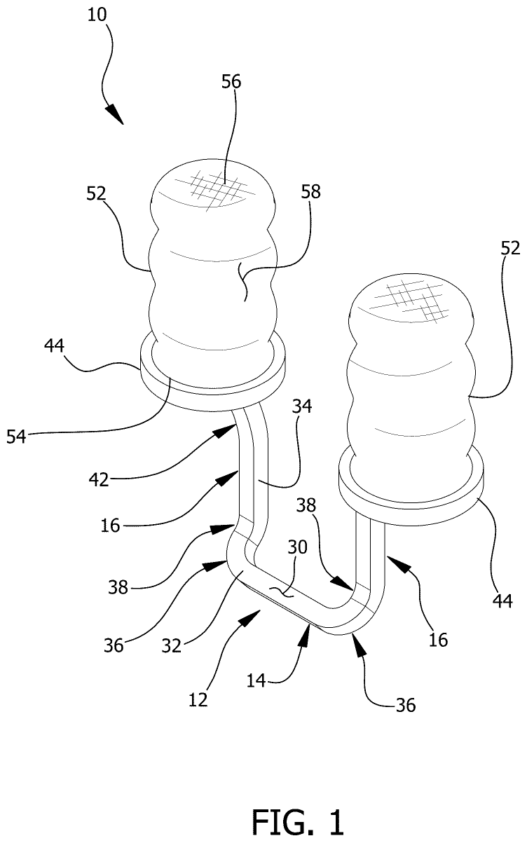

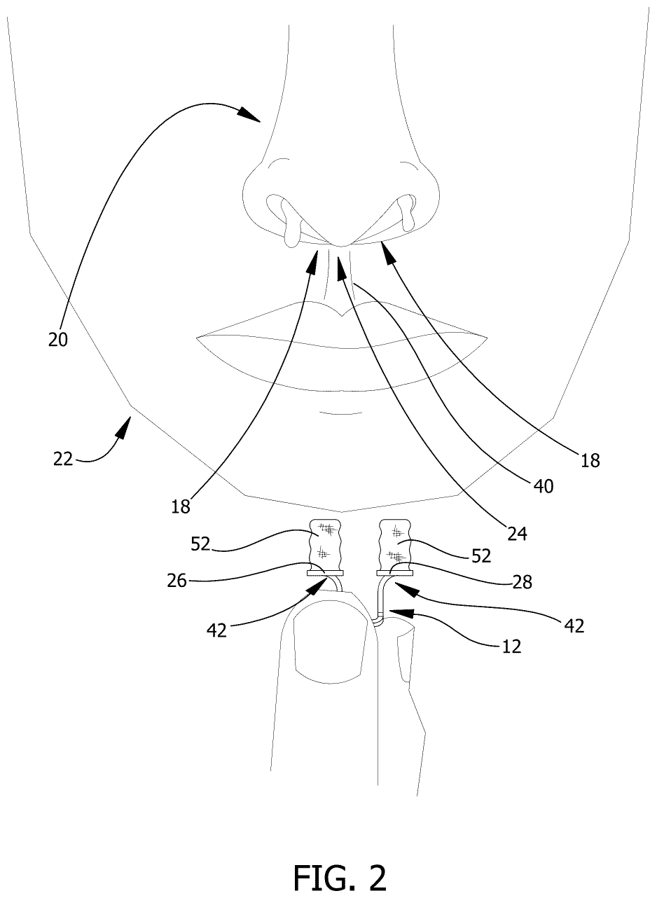

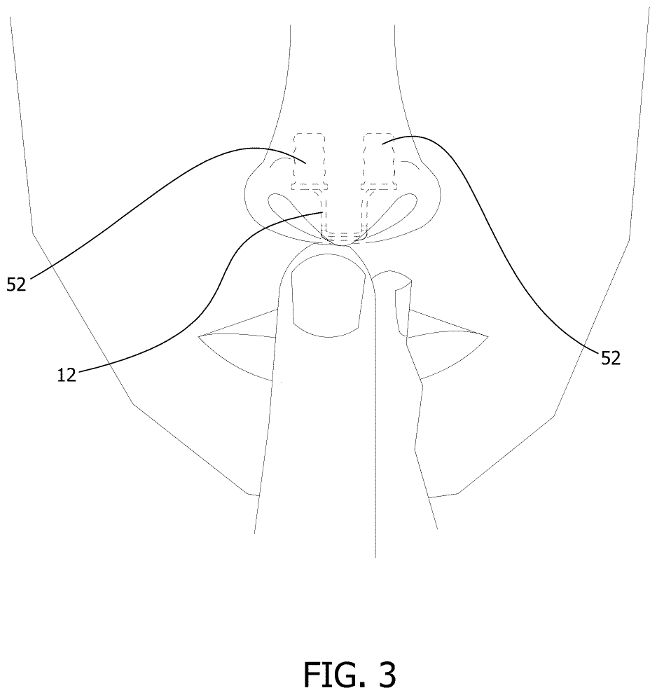

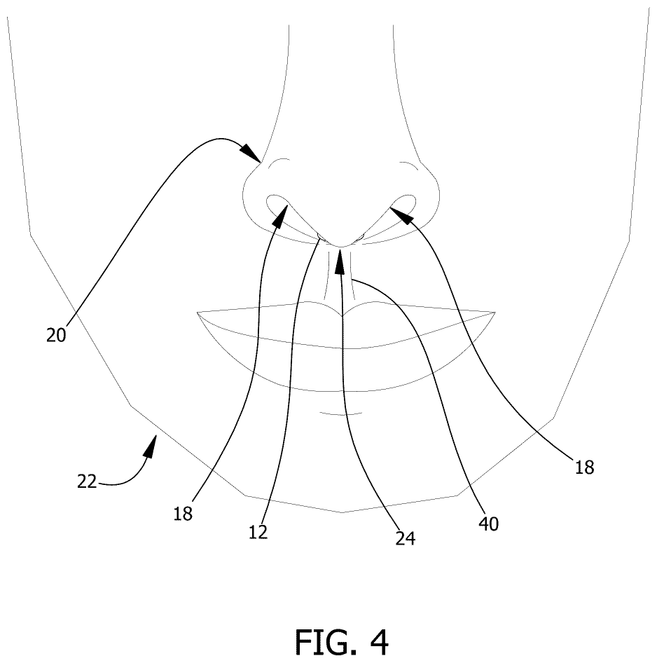

With reference now to the drawings, and in particular to FIGS. 1 through 4 thereof, a new nasal drip device embodying the principles and concepts of an embodiment of the disclosure and generally designated by the reference numeral 10 will be described.

As best illustrated in FIGS. 1 through 4, the nasal drip absorbing device 10 generally comprises a nose clip 12 that has a medial portion 14 extending between and is oriented perpendicular to a pair of leg portions 16 such that the nose clip 12 has a U-shape. Each leg portion 16 of the pair of leg portions 16 of the nose clip 12 can be inserted into a respective nostril 18 of a nose 20 of a user 22 having the medial portion 14 of the nose clip 12 extending across a septum 24 of the nose 20 of the user 22. The nose clip 12 has a first end 26 and a second end 28 and an outer surface 30 extending between the first end 26 and the second end 28. The outer surface 30 has a front side 32 and an inwardly facing side 34; the inwardly facing side 34 lies on a plane that is oriented perpendicular to the front side 32.

The nose clip 12 has a pair of first bends 36 and the pair of first bends 36 is spaced apart from each other. The medial portion 14 of the nose clip 12 is defined between the pair of first bends 36. Each first bend 36 of the pair of first bends 36 is spaced from a respective one of the first end 26 and the second end 28. Additionally, each leg portion 16 of the pair of leg portions 16 is defined between a respective first bend 36 and the respective one of the first end 26 and the second end 28. The nose clip 12 has a pair of second bends 38 and each second bend 38 of the pair of second bends 38 intersects a respective first bend 36. Each second bend 38 of the pair of second bends 38 has a radius of curvature that is offset ninety degrees from a radius of curvature of each first bend 36 of the pair of first bends 36.

The front side 32 of the outer surface 30 corresponding to the medial portion 14 lies on a plane which is oriented at an angle with the front side 32 of the outer surface 30 corresponding to each leg portion 16 of the pair of leg portions 16. Subsequently, the medial portion 14 is spaced from a philtrum 40 of the user 22 when each leg portion 16 of the pair of leg portions 16 is extended into the respective nostril 18 of the nose 20 of the user 22. The nose clip 12 has a pair of third bends 42 and each third bend 42 of the pair of third bends 42 is positioned between a respective second bend 38 and a respective one of the first end 26 and the second end 28. Each third bend 42 of the pair of third bends 42 is positioned closer to the respective one of the first end 26 and the second end 28 than the respective second bend 38.

The inwardly facing side 34 of the outer surface 30 of the nose clip 12 corresponding to each leg portion 16 of the pair of leg portions 16 which extends between a respective second bend 38 and a respective third bend 42 lies on a plane which is oriented parallel to each other. Each leg portion 16 of the pair of leg portions 16 is positioned on opposite sides of the septum 24 of the nose 20 of the user 22 when each leg portion 16 of the pair of leg portions 16 is inserted into the respective nostril 18 of the nose 20 of the user 22. Furthermore, each third bend 42 of the pair of third bends 42 curves in an opposite direction from a respective first bend 36. Subsequently, each of the first end 26 and the second end 28 of the nose clip 12 inhibits touching the septum 24 of the nose 20 of the user 22 when each leg portion 16 of the pair of leg portions 16 is inserted into the respective nostril 18 of the nose 20 of the user 22.

A pair of disks 44 is provided and each disk 44 of the pair of disks 44 has a bottom surface 46 and a top surface 48. The bottom surface 46 of each disk 44 of the pair of disks 44 is coupled to a respective one of the first end 26 and the second end 28 of the nose clip 12. Each disk 44 of the pair of disks 44 closes a respective nostril 18 of the nose 20 of the user 22 when each leg portion 16 of the pair of leg portions 16 is inserted into the respective nostril 18 to inhibit mucous 50 from dripping out of the respective nostril 18.

A pair of cylindrical pads 52 is provided and each cylindrical pad 52 of the pair of cylindrical pads 52 is disposed on a respective one of the leg portions 16 of the pair of leg portions 16 of the nose clip 12. Each cylindrical pad 52 of the pair of cylindrical pads 52 is positioned within a respective nostril 18 of the nose 20 of the user 22 when the pair of leg portions 16 of the nose clip 12 is inserted into the respective nostril 18. Additionally, each cylindrical pad 52 of the pair of cylindrical pads 52 is comprised of a fluid absorbent material, including but not being limited to cotton or rayon, such that each cylindrical pad 52 of the pair of cylindrical pads 52 can absorb the mucous 50 when each cylindrical pad 52 of the pair of cylindrical pads 52 is positioned within the respective nostril 18.

Each cylindrical pad 52 of the pair of cylindrical pads 52 has a bottom end 54 and a top end 56 and an exterior surface 58 extending between the top end 56 and the bottom end 54. The exterior surface 58 of a respective cylindrical pad 52 is continuously arcuate about an axis extending between the top end 56 and the bottom end 54 of the respective cylindrical pad 52 such that each cylindrical pad 52 of the pair of cylindrical pads 52 has a cylindrical shape. Furthermore, each cylindrical pad 52 of the pair of cylindrical pads 52 conforms to a shape of the respective nostril 18. The bottom end 54 of each cylindrical pad 52 of the pair of cylindrical pads 52 is coupled to the top surface 48 of a respective disk 44. Additionally, each cylindrical pad 52 of the pair of cylindrical pads 52 has a diameter that is less than a diameter of the respective disk 44.

In use, the medial portion 14 of the nose clip 12 is gripped and each of the pair of cylindrical pads 52 is inserted into the respective nostril 18 of the nose 20 of the user 22 when the user 22 suffering from a nasal infection, such as the common cold for example. Each of the cylindrical pads 52 absorbs the mucous 50 to inhibit the mucous 50 from dripping from the nose 20 of the user 22. The nose clip 12 and the pair of cylindrical pads 52 are removed from the nose 20 of the user 22 when the pair of cylindrical pads 52 become soaked with the mucous 50 and are subsequently replaced with a new nose clip 12 and pair of cylindrical pads 52.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A nasal drip absorbing device for absorbing mucous that drips from a nose of a user when the user is suffering from a nasal infection, the device comprising:
a nose clip having a medial portion extending between and being oriented perpendicular to a pair of leg portions such that said nose clip has a U-shape wherein each leg portion of said pair of leg portions of said nose clip is configured to be inserted into a respective nostril of the nose of the user having said medial portion of said nose clip extending across a septum of the nose of the user; and
a pair of cylindrical pads, each cylindrical pad of said pair of cylindrical pads being disposed on a respective one of said leg portions of said pair of leg portions of said nose clip wherein each cylindrical pad of said pair of cylindrical pads is configured to be positioned within a respective nostril of the nose of the user when said pair of leg portions of said nose clip is inserted into the respective nostril, each cylindrical pad of said pair of cylindrical pads being comprised of a fluid absorbent material wherein each cylindrical pad of said pair of cylindrical pads is configured to absorb said mucous when each cylindrical pad of said pair of cylindrical pads is positioned within the respective nostril.

2. The nasal drip absorbing device according to claim 1, further comprising:
said nose clip having a first end and a second end and an outer surface extending between said first end and said second end;
said outer surface having a front side and an inwardly facing side;
said inwardly facing side lying on a plane being oriented perpendicular to said front side.

3. The nasal drip absorbing device according to claim 2, further comprising:
said nose clip having a pair of first bends;
said pair of first bends being spaced apart from each other;
said medial portion of said nose clip being defined between said pair of first bends;
each first bend of said pair of first bends being spaced from a respective one of said first end and said second end; and
each leg portion of said pair of leg portions being defined between a respective first bend and said respective one of said first end and said second end.

4. The nasal drip absorbing device according to claim 3, further comprising:
said nose clip having a pair of second bends;
each second bend of said pair of second bends intersecting a respective first bend;
each second bend of said pair of second bends having a radius of curvature being offset ninety degrees from a radius of curvature of each first bend of said pair of first bends.

5. The nasal drip absorbing device according to claim 2, further comprising said front side of said outer surface corresponding to said medial portion lying on a plane being oriented at an angle with said front side of said outer surface corresponding to each leg portion of said pair of leg portions wherein said medial portion is configured to be spaced from a philtrum of the user when each leg portion of said pair of leg portions is extended into the respective nostril of the nose of the user.

6. The nasal drip absorbing device according to claim 4, further comprising:
said nose clip having a pair of third bends;
each third bend of said pair of third bends being positioned between a respective second bend and a respective one of said first end and said second end; and
each third bend of said pair of third bends being positioned closer to said respective one of said first end and said second end than said respective second bend.

7. The nasal drip absorbing device according to claim 6, further comprising said inwardly facing side of said outer surface of said nose clip corresponding to each leg portion of said pair of leg portions which extends between a respective second bend and a respective third bend lying on a plane being oriented parallel to each other wherein each leg portion of said pair of leg portions is configured to be positioned on opposite sides of the septum of the nose of the user when each leg portion of said pair of leg portions is inserted into the respective nostril of the nose of the user.

8. The nasal drip absorbing device according to claim 6, further comprising each third bend of said pair of third bends curving in an opposite direction from a respective first bend wherein each of said first end and said second end of said nose clip is configured to inhibit touching the septum of the nose of the user when each leg portion of said pair of leg portions is inserted into the respective nostril of the nose of the user.

9. The nasal drip absorbing device according to claim 2, further comprising
a pair of disks;
each disk of said pair of disks having a bottom surface and a top surface;
said bottom surface of each disk of said pair of disks being coupled to a respective one of said first end and said second end of said nose clip wherein each disk of said pair of disks is configured to close a respective nostril of the nose of the user when each leg portion of said pair of leg portions is inserted into the respective nostril to inhibit mucous from dripping out of the respective nostril;
each cylindrical pad of said pair of cylindrical pads having a bottom end; and
said bottom end of each cylindrical pad of said pair of cylindrical pads being coupled to said top surface of a respective disk.

10. The nasal drip absorbing device according to claim 9, further comprising each cylindrical pad of said pair of cylindrical pads having a diameter being less than a diameter of said respective disk.

11. The nasal drip absorbing device according to claim 1, further comprising:
each cylindrical pad of said pair of cylindrical pads having a bottom end and a top end and an exterior surface extending between said top end and said bottom end; and
said exterior surface of a respective cylindrical pad being continuously arcuate about an axis extending between said top end and said bottom end of said respective cylindrical pad such that each cylindrical pad of said pair of cylindrical pads has a cylindrical shape wherein each cylindrical pad of said pair of cylindrical pads is configured to conform to a shape of the respective nostril.

12. A nasal drip absorbing device for absorbing mucous that drips from a nose of a user when the user is suffering from a nasal infection, the device comprising:
a nose clip having a medial portion extending between and being oriented perpendicular to a pair of leg portions such that said nose clip has a U-shape wherein each leg portion of said pair of leg portions of said nose clip is configured to be inserted into a respective nostril of the nose of the user having said medial portion of said nose clip extending across a septum of the nose of the user, said nose clip having a first end and a second end and an outer surface extending between said first end and said second end, said outer surface having a front side and an inwardly facing side, said inwardly facing side lying on a plane being oriented perpendicular to said front side, said nose clip having a pair of first bends, said pair of first bends being spaced apart from each other, said medial portion of said nose clip being defined between said pair of first bends, each first bend of said pair of first bends being spaced from a respective one of said first end and said second end, each leg portion of said pair of leg portions being defined between a respective first bend and said respective one of said first end and said second end, said nose clip having a pair of second bends, each second bend of said pair of second bends intersecting a respective first bend, each second bend of said pair of second bends having a radius of curvature being offset ninety degrees from a radius of curvature of each first bend of said pair of first bends, said front side of said outer surface corresponding to said medial portion lying on a plane being oriented at an angle with said front side of said outer surface corresponding to each leg portion of said pair of leg portions wherein said medial portion is configured to be spaced from a philtrum of the user when each leg portion of said pair of leg portions is extended into the respective nostril of the nose of the user, said nose clip having a pair of third bends, each third bend of said pair of third bends being positioned between a respective second bend and a respective one of said first end and said second end, each third bend of said pair of third bends being positioned closer to said respective one of said first end and said second end than said respective second bend, said inwardly facing side of said outer surface of said nose clip corresponding to each leg portion of said pair of leg portions which extends between a respective second bend and a respective third bend lying on a plane being oriented parallel to each other wherein each leg portion of said pair of leg portions is configured to be positioned on opposite sides of the septum of the nose of the user when each leg portion of said pair of leg portions is inserted into the respective nostril of the nose of the user, each third bend of said pair of third bends curving in an opposite direction from a respective first bend wherein each of said first end and said second end of said nose clip is configured to inhibit touching the septum of the nose of the user when each leg portion of said pair of leg portions is inserted into the respective nostril of the nose of the user;
a pair of disks, each disk of said pair of disks having a bottom surface and a top surface, said bottom surface of each disk of said pair of disks being coupled to a respective one of said first end and said second end of said nose clip wherein each disk of said pair of disks is configured to close a respective nostril of the nose of the user when each leg portion of said pair of leg portions is inserted into the respective nostril to inhibit mucous from dripping out of the respective nostril; and
a pair of cylindrical pads, each cylindrical pad of said pair of cylindrical pads being disposed on a respective one of said leg portions of said pair of leg portions of said nose clip wherein each cylindrical pad of said pair of cylindrical pads is configured to be positioned within a respective nostril of the nose of the user when said pair of leg portions of said nose clip is inserted into the respective nostril, each cylindrical pad of said pair of cylindrical pads being comprised of a fluid absorbent material wherein each cylindrical pad of said pair of cylindrical pads is configured to absorb the mucous when each cylindrical pad of said pair of cylindrical pads is positioned within the respective nostril, each cylindrical pad of said pair of cylindrical pads having a bottom end and a top end and an exterior surface extending between said top end and said bottom end, said exterior surface of a respective cylindrical pad being continuously arcuate about an axis extending between said top end and said bottom end of said respective cylindrical pad such that each cylindrical pad of said pair of cylindrical pads has a cylindrical shape wherein each cylindrical pad of said pair of cylindrical pads is configured to conform to a shape of the respective nostril, said bottom end of each cylindrical pad of said pair of cylindrical pads being coupled to said top surface of a respective disk, each cylindrical pad of said pair of cylindrical pads having a diameter being less than a diameter of said respective disk.
